(19) United States
(12) Patent Application Publication
(10) Pub. No.: US 2026/0218111 A1
(43) Pub. Date: Jul. 30, 2026

(54) SPERM SORTING DEVICE AND SPERM SORTING METHOD

(71) Applicant: GREENISH INC., Cheongju-si Chungcheongbuk-do (KR)

(72) Inventor: In Sung SUNG, Goyang-si Gyeonggi-do (KR)

(21) Appl. No.: 19/152,003

(22) PCT Filed: Oct. 23, 2023
(86) PCT No.: PCT/KR2023/016495

§ 371 Date: Jul. 30, 2025

(30) Foreign Application Priority Data
Feb. 6, 2023 (KR) 10-2023-0015456

(51) Int. Cl. C12M 1/00; C12N 5/071; C12N 5/076

(52) U.S. Cl. CPC C12M 47/04; C12N 5/061; C12N 5/0612

(57) ABSTRACT

Provided are a device for selecting sperm with excellent motility in a simple manner and a method thereof. The device for sorting sperm includes: a flow part configured to form an inclination, wherein the flow part includes at least two grooves spaced apart from each other along a direction of the inclination.

### TECHNICAL FIELD

The present invention relates to a device for sorting sperm. More particularly, the present invention relates to a device for selecting sperm with excellent motility in a simple manner and a method thereof.

### BACKGROUND ART

In general, subfertility or sterility is academically defined as the inability of a man and woman of reproductive age to achieve pregnancy within one year despite having regular unprotected sexual intercourse. Conventionally, the term “sterility” has also been used to refer to a state in which pregnancy is impossible; however, when the cause of infertility is identified, it is referred to as sterility, and when the cause is unknown, it is referred to as subfertility. In this specification, the terms subfertility and sterility may be used interchangeably.

It has been reported that approximately 15% of couples of reproductive age experience subfertility or sterility. When the cause of infertility lies with the male partner, it is referred to as male infertility, which is estimated to account for about 40% to 50% of all infertile couples. The causes of male infertility are also diverse, but they may be broadly classified into sperm abnormalities, obstruction of the sperm transport pathway, semen abnormalities, sexual dysfunction, and other causes such as chronic diseases. Among these, sperm abnormalities include issues with the sperm itself (e.g., quantity, morphology, motility, volume, pH, etc.), oligospermia, and asthenozoospermia.

### DISCLOSURE

### Technical Problem

In the case of male infertility, particularly due to sperm abnormalities, achieving fertilization through conventional methods is difficult. For this reason, assisted reproductive technologies (ART), such as intrauterine insemination (IUI) or in vitro fertilization-embryo transfer (IVF-ET), are performed to overcome subfertility or infertility. These are methods of inducing pregnancy by injecting sperm collected from a male into the uterine cavity, or by fertilizing an egg in vitro and then implanting the embryo into the uterus. In cases of male infertility, sperm with excellent motility and high quality are selected to improve the chances of pregnancy.

Meanwhile, the criteria for normal semen parameters suggested by the World Health Organization are approximately 15 million sperm per mL, ≥40% motile sperm, ≥58% live sperm, and ≥4% sperm with normal morphology. That is, even according to these criteria, selecting normal sperm from among the numerous sperm contained in semen is never an easy task, and it is even more difficult to select high-quality sperm from the semen of a male diagnosed with sperm abnormalities. For this reason, sperm selection for assisted reproductive technology is, in reality, carried out based on the examiner's intuition rather than according to clear standards.

Therefore, the present invention has been made in view of the above problems, and it is one object of the present invention to provide a device for selecting statistically significant high-quality sperm, particularly sperm having motility, by using a relatively simple method.

It is another object of the present invention to provide a method of selecting statistically significant high-quality sperm, particularly sperm having high motility, through a relatively simple process.

The objects of the present invention are not limited to the technical problems mentioned above, and other technical problems not specifically mentioned may be clearly understood by those skilled in the art from the following description.

### Technical Solution

In accordance with an aspect of the present invention, the above and other objects can be accomplished by the provision of a device for sorting sperm, including: a flow part configured to form an inclination, wherein the flow part includes at least two grooves spaced apart from each other along a direction of the inclination.

The device for sorting sperm may be configured such that a liquid containing sperm flows along the flow part in an inclined direction.

In accordance with another aspect of the present invention, provided is a method of sorting sperm, the method including: loading a sperm-containing liquid at a position on a flow part configured to form an inclination and provided with at least two grooves spaced apart from each other along a direction of the inclination; and collecting only a liquid containing sperm that has flowed along the flow part and pooled in one or more of the plural grooves.

The collecting may include: collecting the liquid in a groove that is not the most adjacent to the loading position of the liquid.

The liquid may flow along the flow part naturally by gravity without the application of an external force.

The viscosity of the sperm-containing liquid to be loaded may be prepared by mixing semen with one or more of physiological saline and a PBS buffer solution.

Specific details of other embodiments are included in the detailed description.

### Advantageous Effects

According to embodiments of the present invention, a group of sperm exhibiting relatively excellent motility can be selected simply by allowing semen collected from a male to flow as it is, or in combination with a liquid substance.

Through this, it is expected that the success rate of assisted reproductive technology for humans or animals can be increased.

The effects of the embodiments of the present invention are not limited to those illustrated above, and various additional effects are included within the scope of this specification.

### DESCRIPTION OF DRAWINGS

FIG. 1 illustrates the perspective view of a device for sorting sperm according to an embodiment of the present invention.

FIG. 2 illustrates the side view of the device for sorting sperm of FIG. 1.

FIG. 3 illustrates the schematic diagram of a process of sorting sperm using the device for sorting sperm shown in FIG. 2.

FIG. 4 illustrates the side view of a device for sorting sperm according to another embodiment of the present invention.

FIG. 5 illustrates the side view of a device for sorting sperm according to still another embodiment of the present invention.

FIG. 6 illustrates the side view of the device for sorting sperm according to still another embodiment of the present invention.

FIG. 7 illustrates the side view of the device for sorting sperm according to still another embodiment of the present invention.

### BEST MODEL

Advantages and features of the present invention and methods of accomplishing the same may be understood more readily by reference to the following detailed description of preferred embodiments and the accompanying drawings. The present invention may, however, be embodied in many different forms and should not be construed as being limited to the embodiments set forth herein. Rather, these embodiments are provided so that this invention will be thorough and complete and will fully convey the concept of the invention to those skilled in the art, and the present invention will only be defined by the appended claims.

Also, the scope of claims is not a matter explaining the technical content that constitutes the substance of the disclosure, but a matter indicating the scope of rights claimed based on the technical configuration disclosed by the detailed description of the disclosure. Therefore, it is inevitable to some extent that the claims are composed of abstract superordinate concepts that include the technology disclosed in the detailed description of the disclosure, and if a person skilled in the art can understand the technical composition or combination and effect of the claims throughout the entire specification, the claims should be considered to be supported by the detailed description of the disclosure.

That is, various changes may be made to embodiments presented in the present disclosure. Examples described below are not intended to limit embodiments of the present disclosure, and should be understood to include all modifications, equivalents, or alternatives thereto.

If any term described in this specification is intended to be used as a specific meaning, its meaning may be defined and used and should be interpreted accordingly. Unless defined otherwise, all technical and scientific terms used herein have the same meaning as commonly understood by one of ordinary skill in the art to which this invention belongs. Further, unless defined otherwise, all terms defined in generally used dictionaries may not be overly interpreted.

As used herein, the term “and/or” includes any and all combinations of one or more of the associated listed items. As used herein, the singular forms “a”, “an” and “the” are intended to include the plural forms as well, unless the context clearly indicates otherwise. It will be further understood that the terms “comprises” and/or “comprising,” when used in this specification, specify the presence of stated components, but do not preclude the presence or addition of one or more other components. A numerical range expressed using ‘to’ indicates a numerical range including values stated before and after ‘to’ as the lower and upper limits. A numerical range expressed using ‘about’ or ‘approximately’ indicates a value or a numerical range within 20% of the value or the numerical range stated after ‘about’ or ‘approximately’.

In this specification, ordinal modifiers such as ‘first component’, ‘second component’, ‘first-first component’, etc., when referring to components, are only used to distinguish one component from another. Therefore, the first component referred to below may be referred to as the second component within the scope of the technical idea of the present disclosure. For example, what is referred to as the first component in one embodiment may be referred to as the second component in another embodiment. In addition, it is obvious that what is referred to as the first component in the detailed description may be referred to as the second component in the claims.

The first direction X means any direction on the plane, and the second direction Y means another direction intersecting or orthogonal to the first direction X within the plane. The third direction Z means another direction intersecting or perpendicular to the plane.

In the drawings, components may be enlarged or reduced in size, thickness, width, length, and the like for convenience and clarity of description, and thus the present invention is not limited to the illustrated form.

Spatially relative terms, such as “above,” “upper,” “on,” “below,” “beneath,” “lower,” and the like, may be used herein for ease of description to describe one element or feature's relationship to another element(s) or feature(s) as illustrated in the figures. It will be understood that the spatially relative terms are intended to encompass different orientations of the device in use in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below.

In this specification, the term “most adjacent” refers to the one that is closest or most neighboring among a plurality of components that are arranged repeatedly in at least one direction. For example, when there is no component between a plurality of adjacent components that may be referred to or classified as being substantially identical to the components, the plural components may be understood to be most adjacent to each other.

In this specification, Computer-Assisted Sperm Analysis (CASA) refers to one method of semen analysis. CASA may be performed by a known method. The characteristics of sperm described below may refer to values measured by CASA, but it is to be understood that they also include values measured by other methods, as long as reliability is maintained.

The sperm characteristics described below, particularly motility and amplitude of lateral head displacement, are known to have a significant impact on fertilization rates.

Motility (MOT) of sperm refers to the percentage of sperm that are moving among those included in a sample. The unit of sperm motility may be percent (%).

Curvilinear velocity (VCL) of sperm refers to the velocity measured by tracking the random motion or movement of sperm. The unit of curvilinear velocity may be micrometers per second (μm/sec).

Straight line velocity (VSL) of sperm refers to the velocity measured along the shortest straight-line distance from one point to another. The unit of straight line velocity may be micrometers per second (μm/sec).

Average path velocity (VAP) of sperm refers to the velocity measured over subdivided segments of a path taken by sperm from one point to another. The unit of average path velocity may be micrometers per second (μm/sec).

Linearity (LIN) of sperm may refer to the ratio of VSL to VCL (VSL/VCL). Additionally, straightness (STR) of sperm may refer to the ratio of VSL to VAP (VSL/VAP).

Amplitude of lateral head displacement (ALH) of sperm refers to the amplitude magnitude of the lateral movement of the sperm head. The unit of ALH may be micrometers (μm).

Beat cross frequency (BCF) of sperm refers to the frequency of the sperm head's oscillatory movement. Concentration or count of sperm refers to the number of sperm contained in 1 mL of semen. The unit of sperm concentration may be number/milliliter (count/mL). Strict morphology of sperm refers to the percentage of sperm with normal morphology. The unit of sperm morphology may be percent (%).

The aforementioned sperm characteristics may be understood as conventional parameters in the technical field, and further detailed descriptions are omitted.

Hereinafter, the present invention will be described in detail with reference to the accompanying drawings.

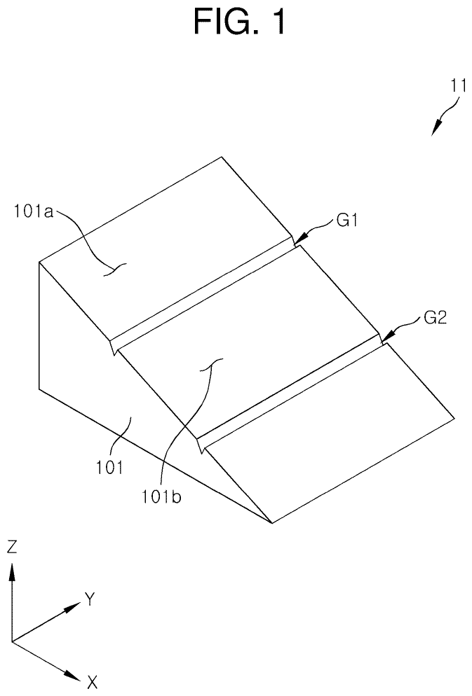

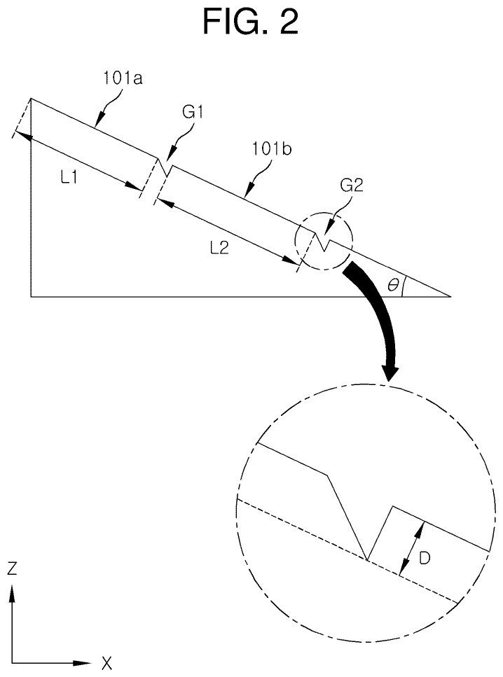

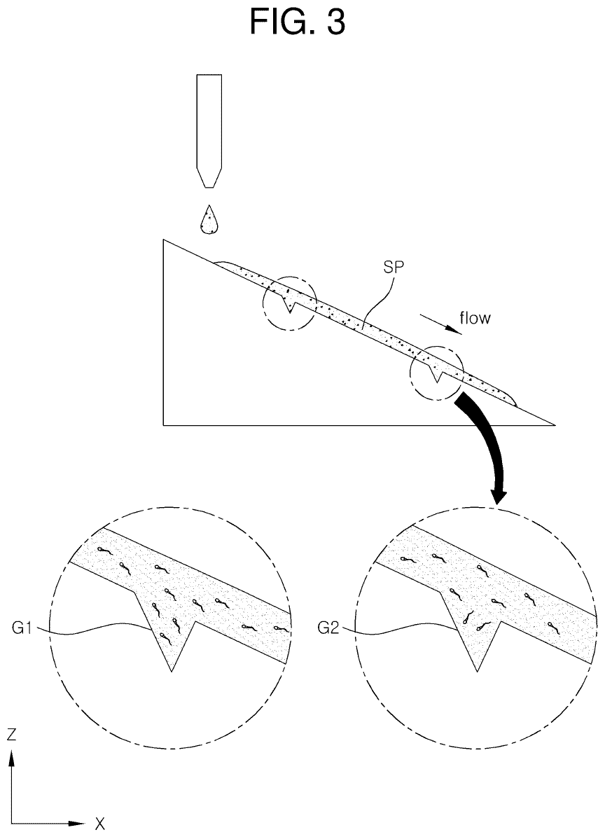

FIG. 1 illustrates the perspective view of a device for sorting sperm according to an embodiment of the present invention. FIG. 2 illustrates the side view of the device for sorting sperm of FIG. 1. FIG. 3 illustrates the schematic diagram of a process of sorting sperm using the device for sorting sperm shown in FIG. 2.

Referring to FIGS. 1 to 3, a device 11 for sorting sperm according to the embodiment may include a flow part 101. The device 11 for sorting sperm according to the present invention, and a sperm sorting method to be described below may be intended to select, classify, or separate high-quality sperm from among those contained in semen of a human or a non-human animal. In particular, it may be intended to sort a group of sperm having, or likely to have, excellent motility (MOT) and amplitude of lateral head displacement (ALH).

Although not illustrated in the drawings, the device for sorting sperm 11 according to the embodiment may be implemented as a fluidic chip, a lab-on-a-chip, or a microchip. For this purpose, the device for sorting sperm 11 may further include a cover (not shown), a liquid injection port formed in the cover, and the like.

The flow part 101 (or the inclined part) may provide a space in which loaded sperm or a liquid containing sperm, such as semen or a liquid containing semen (hereinafter referred to as “loading liquid”), may flow by gravity without the application of external force. An upper surface of the flow part 101 may at least partially form an inclined surface that is sloped with respect to a horizontal plane, for example, in a direction including a first direction X and a second direction Y. Specifically, from a side view including the first direction X and a third direction Z, the upper surface of the flow part 101 may provide a flow path extending in a direction intersecting the first direction X and the third direction Z.

An inclination angle θ formed by the flow part 101 may be in the range of 10° to 50°, or about 15° to about 40°. FIG. 2 illustrates an embodiment in which the flow part 101 has an upper surface with a constant inclination angle along the inclined direction. In another embodiment, the upper surface of the flow part 101 may have a varying inclination angle along the inclined direction. In such a case, the varying inclination angles may all fall within the aforementioned range. If the inclination angle is too steep, it may fail to effectively select high-quality sperm. Conversely, if the inclination angle is too gentle, it may also fail to meaningfully select high-quality sperm.

The material of the flow part 101 is not particularly limited, but, for example, it may be made of a material that has excellent flow characteristics and low liquid permeability, while not affecting the integrity, viability, or survival of the sperm, such as a plastic synthetic resin. However, the present invention is not limited thereto.

The flow part 101, and more specifically, the upper surface (or inclined surface, or flow surface) of the flow part 101, may have a plurality of grooves G1 and G2 (also referred to as recessed portions or a collection part). The plural grooves G1 and G2 may be spaced apart from each other along the inclined direction provided by the upper surface of the flow part 101, i.e., along the flow direction of the loading liquid.

FIG. 1 illustrates a case in which the grooves G1 and G2 both extend in the second direction Y and are exposed at the edge of the flow part 101 in the second direction Y, but the present invention is not limited thereto. So long as the grooves G1 and G2 are positioned along a flow path where the loading liquid SP flows down the inclined surface, their shapes are not particularly limited.

The plural grooves G1 and G2 may include a first groove G1 and a second groove G2. Although FIG. 1 illustrates a case in which the grooves G1 and G2 are provided as two grooves, in other embodiments, the grooves G1 and G2 may be provided as two or more. Along the inclined direction formed by the flow part 101, the first groove G1 and the second groove G2 may divide the upper surface of the flow part 101 into a first section 101a (also referred to as a first inclined part, a first inclined surface, a first flow part, or a first flow surface) and a second section 101b.

The first section 101a refers to a region located above the first groove G1 in the inclined direction, or upstream in the direction of gravity. The second section 101b refers to the region between the first groove G1 and the second groove G2, i.e., a region located downstream of the first groove G1 and upstream of the second groove G2 in the inclined direction. The inclination angles (θ) of the first section 101a and the second section 101b may be substantially the same or within a ±5% range. A user may load a loading liquid SP at any position of the first section 101a. That is, a loading part or a dropping part may be formed at any position of the first section 101a. The loading liquid SP flowing along the flow part 101 may flow down along the inclined direction, pass through the first groove G1, continue along the second section 101b, and reach the second groove G2. A portion of the loading liquid SP may pool in the first groove G1 and the second groove G2. Furthermore, the loading liquid SP may, of course, continue to flow further downstream beyond the second groove G2 in the inclined direction.

A first length L1 of the first section 101a in an inclined direction (or a first liquid flow distance) and a second length L2 of the second section 101b in an inclined direction (or a second liquid flow distance) may fall within a predetermined range. Here, the second length L2 may refer to a separation distance between the first groove G1 and the second groove G2 in the inclined direction.

In an exemplary embodiment, the first length L1 may be in the range of about 2.0 cm to 5.0 cm, and the second length L2 may be in the range of about 2.0 cm to 5.0 cm. When the first length L1 and the second length L2, particularly the second length L2, fall within the above range, high-quality sperm may be meaningfully selected.

A depth D of the first groove G1 and the second groove G2 may fall within a predetermined range. Here, the depth of the grooves refers to the maximum length in the normal direction of the inclined surface, measured from the inclined surface before or after the groove to a certain point within the groove. In an exemplary embodiment, the depth D of the first groove G1 and the second groove G2 may be in the range of about 0.2 cm to 1.0 cm. If the depth D of the first groove G1 and/or the second groove G2 is greater than the above range, it may fail to meaningfully distinguish differences in characteristics such as motility and/or the amplitude of lateral head displacement.

The device 11 for sorting sperm described above may utilize the rheotactic property of sperm—wherein sperm swim against the direction of fluid flow—to simply and easily select sperm with excellent motility and the amplitude of lateral head displacement. In an exemplary embodiment, the sperm sorting method may include loading the loading liquid SP at a loading part, for example, at a certain position on the first section 101a, and collecting the liquid that accumulates in one or more of the plural grooves G1 and G2 as the loading liquid SP flows along the flow part 101. Here, it is of course understood that the liquid collected in the grooves G1 and G2 contains sperm. The inventors of the present invention have confirmed that there is a meaningful difference in the characteristics of sperm contained in the liquids collected in the grooves G1 and G2, which are spaced apart from each other, and have thereby completed the present invention. Specifically, sperm contained in the liquid pooled in a groove not most adjacent to the loading part of the loading liquid SP, for example, the second groove G2, may have higher characteristics such as motility than sperm contained in the liquid pooled in the first groove G1, which is most adjacent to the loading part. Accordingly, by selectively collecting only the liquid pooled in a groove not most adjacent to the loading part, such as the second groove G2, rather than the first groove G1 that is most adjacent, high-quality sperm may be effectively selected.

For meaningful selection of high-quality sperm, factors such as the angle of the flow part 101 of the device 11 for sorting sperm and the positions of the grooves G1 and G2 may be important. In some embodiments, the sperm sorting method according to the present embodiment may control flow characteristics by adjusting the viscosity of the loading liquid SP. For this purpose, a mixture of test semen with one or more of physiological saline, PBS buffer solution, and other liquids may be used as the loading liquid SP.

Hereinafter, other embodiments of the present invention will be described. However, descriptions of configurations that are substantially the same as or very similar to the above-described embodiments will be omitted, as they will be clearly understood by those of ordinary skill in the art from the accompanying drawings. In addition, this specification implies the disclosure of combinations between the technical ideas disclosed in different embodiments, unless such combinations conflict with one another.

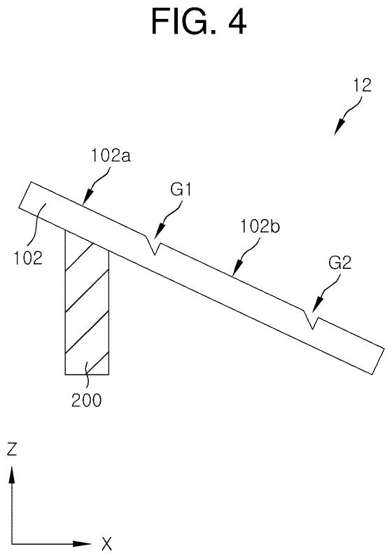

FIG. 4 illustrates the side view of a device for sorting sperm according to another embodiment of the present invention, showing the same position as in FIG. 2.

Referring to FIG. 4, a device 12 for sorting sperm according to the embodiment includes a flow part 102, and differs from the previously described embodiment in that it further includes a support part 200.

In the previously described embodiment, the flow part had a wedge or triangular shape from a side view, providing an inclined surface for the loading liquid to flow while simultaneously contacting the ground. However, in the present embodiment, the device 12 for sorting sperm may configure the flow part 102 in a substantially plate-like shape, and be inclined by using the support 200. In other words, the support 200 may support the flow part 102 in such a way that one side and the other side of the flow part 102 are positioned at different heights, thereby spacing the flow part 102 apart from the ground. The lower surface of the plate-shaped flow part 102 may have a structure that is connectable to the support 200, and the upper surface of the flow part 102 forms an inclined surface (or fluid flow surface), and includes multiple sections such as the first groove G1, the second groove G2, and a first section 102a and a second section 102b, which are divided by the grooves G1 and G2, as previously described.

Although not illustrated in the drawings, the support 200 may further include means for adjusting the angle of the flow part 102. The method of adjusting the angle is not particularly limited, but may be implemented by, for example, adjusting and/or changing the length of the support 200 in the third direction Z, and/or adjusting and/or changing the coupling position between the support 200 and the flow part 102.

The device 12 for sorting sperm according to the present embodiment may include the flow part 102 and the support 200 that are configured to be reversibly coupled and detachable from each other. Through this configuration, the volume of the device 12 for sorting sperm may be reduced, allowing it to be provided as a kit, for example, in a form similar to a lab-on-a-chip.

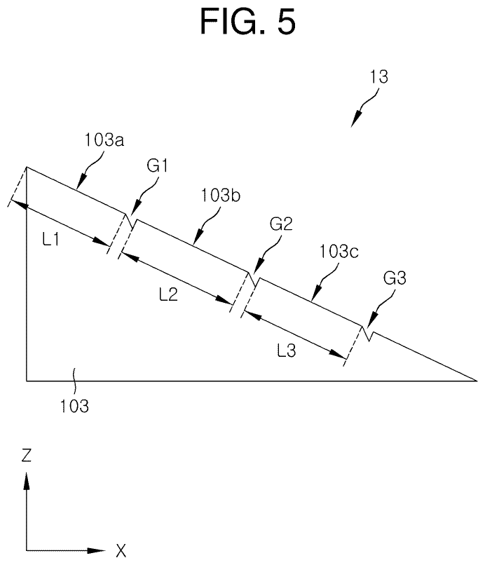

FIG. 5 illustrates the side view of a device for sorting sperm according to still another embodiment of the present invention, showing the same position as in FIG. 2.

Referring to FIG. 5, a device for sorting sperm 13 according to the present embodiment includes a flow part 103 in which a plurality of grooves G1, G2 and G3 are formed, and differs from the previously described embodiments in that three or more grooves are provided. Although not illustrated in the drawing, as previously described, the flow part may be provided in a plate-like shape, and the device for sorting sperm may further include a support.

The flow part 103 may provide a space through which the loading liquid may flow by gravity. Specifically, the upper surface of the flow part 103 may provide a flow path extending in a direction intersecting the first direction X and the third direction Z.

The upper surface of the flow part 103 may have a first groove G1, a second groove G2, and a third groove G3. The first groove G1 to the third groove G3 may be spaced apart from each other in an inclined direction. In addition, the first groove G1 to the third groove G3 may divide the upper surface of the flow part 103 into a first section 103a, a second section 103b, and a third section 103c. The first section 103a refers to a region located upstream of the first groove G1 in the inclined direction. The second section 103b refers to a region between the first groove G1 and the second groove G2. The third section 103c refers to a region between the second groove G2 and the third groove G3. The inclination angles of the first section 103a to the third section 103c may be substantially the same.

For sperm sorting, the loading liquid may be loaded at any position of the first section 103a. Then, as the loading liquid flows down along the flow part 103, it may pass through the first groove G1, continue along the second section 103b, and reach the second groove G2. Subsequently, the loading liquid may continue flowing, pass through the second groove G2, flow along the third section 103c, and reach the third groove G3. A portion of the loading liquid may pool in each of the first groove G1 to the third groove G3. Furthermore, the loading liquid may, of course, flow further downstream beyond the third groove G3 in the inclined direction.

A first length L1 and a second length L2 may each be in the range of about 2.0 cm to 5.0 cm. In addition, a third length L3 may also be in the range of about 2.0 cm to 5.0 cm. In some embodiments, the second length L2 may be greater than the third length L3. The ratio of the second length L2 to the third length L3 may be in the range of greater than about 1.0 and 2.0 or less, about 1.1 to about 1.8, or about 1.2 to about 1.6. When the values and ratios of the second length L2 and the third length L3 fall within the above ranges, the characteristics of sperm collected in the second groove G2 and the third groove G3 may be meaningfully distinguished. Since the depth of each of the grooves G1, G2, and G3 has been previously described, overlapping explanations are omitted.

The device for sorting sperm 13 according to the present embodiment may also be used in such a way that a user loads a loading liquid at any position of the first section 103a, and after the loading liquid flows along the flow part 103 and pools in the plural grooves G1, G2, and G3, the user collects the liquid pooled in a selected groove among the grooves G1, G2, and G3. Specifically, instead of the first groove G1, which is most adjacent to the loading part of the loading liquid, or the third groove G3, which is the farthest from the loading part, sperm contained in the liquid pooled in a non-most-adjacent groove, such as the second groove G2, may have higher characteristics such as higher motility compared to sperm contained in the liquid pooled in the first groove G1 and/or the third groove G3.

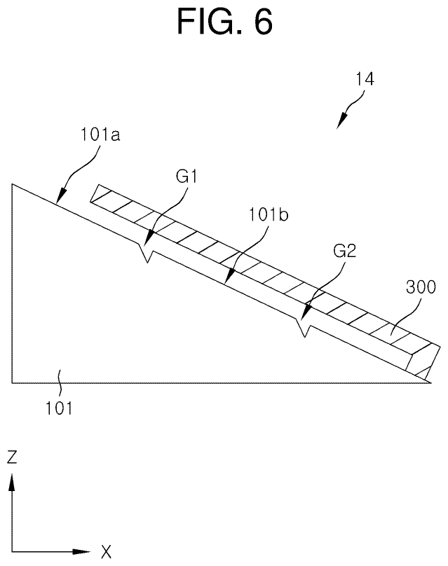

FIG. 6 illustrates the side view of the device for sorting sperm according to still another embodiment of the present invention, showing the same position as in FIG. 2.

Referring to FIG. 6, a device 14 for sorting sperm according to the present embodiment includes the flow part 101, and differs from the previously described embodiments in that it further includes a cover 300. Although not illustrated in the drawing, as previously described, a flow part may be provided in a plate-like shape, and the device for sorting sperm may further include a support.

As previously described, the loaded liquid may flow along the upper surface of the flow part 101 and may pool in the first groove G1 and the second groove G2. Here, if the amount of loading liquid is too large, the liquid may not form a thin film that flows while in contact with the upper surface of the flow part 101; instead, the upper layer of the loading liquid may flow over the lower layer without fully contacting the surface. In such a case, despite the rheotactic behavior of sperm, it may be difficult to meaningfully distinguish the characteristics of sperm contained in the liquid pooled in the first groove G1 and the second groove G2.

To address this, the cover 300 according to the present embodiment may cover the upper surface of the flow part 101, and more specifically, the upper regions of the first groove G1 and the second groove G2, thereby forming a flow space for the liquid between the cover 300 and the flow part 101. For example, when an excessive amount of loading liquid is introduced, the upper layer of the liquid may be allowed to overflow above the cover 300 and be discarded, while the lower layer of the loading liquid, which flows in substantial contact with the flow part 101, may flow through the space between the cover 300 and the flow part 101 and pool in the first groove G1 and the second groove G2.

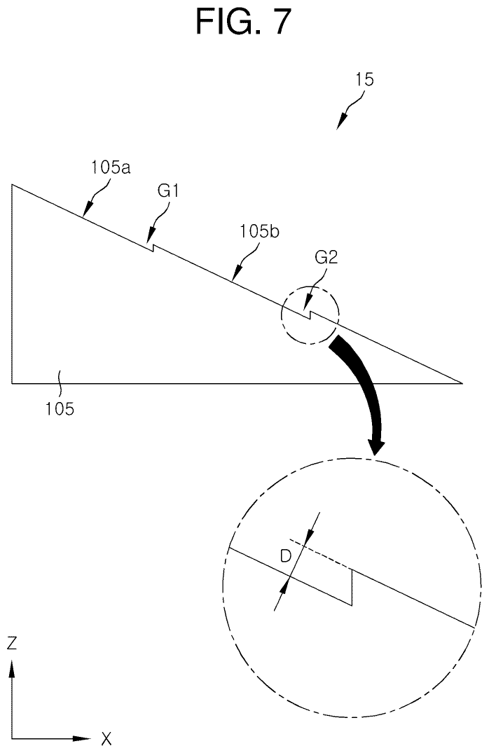

FIG. 7 illustrates the side view of the device for sorting sperm according to still another embodiment of the present invention, showing the same position as in FIG. 2.

Referring to FIG. 7, a device 15 for sorting sperm according to the present embodiment includes a flow part 105 having a first groove G1 and a second groove G2, and differs from the previously described embodiments in that a first section 105a and a second section 105b of the flow part 105 have substantially the same inclination angle, or an inclination angle within a ±5% range of each other, but are not directly connected and instead form a height difference between them. Although not illustrated in the drawing, as previously described, the flow part may be provided in a plate-like shape, and the device for sorting sperm may further include a support.

In an exemplary embodiment, the lower end of the first section 105a may be positioned lower in a third direction Z than the upper end of the second section 105b. The first groove G1 may be formed by a step created between the lower end of the first section 105a and the upper end of the second section 105b.

Likewise, the lower end of the second section 105b may be positioned lower in the third direction Z than the upper end of a third section (for example, an inclined surface located downstream of the second groove G2 in the inclined direction). The second groove G2 may be formed by a step created between the lower end of the second section 105b and the upper end of the third section.

As described above, the depth D of the grooves G1 and G2 may be defined as the maximum length in the normal direction to the inclined surface, measured from the inclined surface adjacent to the groove to the position of the groove.

Hereinafter, the present invention will be described in more detail with reference to experimental examples of the present invention.

### EXAMPLE 1

A device for sorting sperm including a flow part having the shape shown in FIG. 1 was fabricated. The flow part was manufactured using acrylate. The lengths of the first section and second section were set to about 4.0 cm. The depths of the first groove and second groove were set to about 0.5 cm. The inclination angle of the flow part was set to about 25°. Then, 5 cc of thawed bovine semen was dropped and loaded onto the first section. The loading position was the uppermost point of the first section. After 10 minutes had elapsed following the dropwise application, the liquid pooled in the first groove and second groove was collected, and the characteristics of the sperm were analyzed using CASA. The experiment was repeated five times.

### EXAMPLE 2

A device for sorting sperm including a flow part having the shape shown in FIG. 5 was fabricated. The length of the third section was set to about 4.0 cm, and the depth of the third groove was set to about 0.5 cm. Other conditions were the same as in Example 1 for fabrication and analysis.

### EXAMPLE 3

Except that the depth of the grooves was set to 0.1 cm, the device was fabricated and analyzed in the same manner as in Example 1.

### EXAMPLE 4

Except that the lengths of the first section and second section were set to 1.0 cm, the device was fabricated and analyzed in the same manner as in Example 1.

### EXAMPLE 5

Except that the lengths of the first section and second section were set to 8.0 cm, the device was fabricated and analyzed in the same manner as in Example 1.

### EXAMPLE 6

Except that the inclination angle of the flow part was set to 60°, the device was fabricated and analyzed in the same manner as in Example 1.

Each of Examples 1 to 6 was conducted five times. The liquids pooled in the first groove and second groove (or third groove) were analyzed using CASA, and the results were averaged and shown in Tables 1 and 2 below.

TABLE 1

| | Example 1 | | Example 2 | | |
|---|---|---|---|---|---|
| | a first groove | a second groove | a first groove | a second groove | a third groove |
| MOT | 56.69 | 79.76 | 55.77 | 80.45 | 67.52 |
| VCL | 55.76 | 80.42 | 56.45 | 80.25 | 78.15 |
| VAP | 31.79 | 43.61 | 31.54 | 43.33 | 35.13 |
| VSL | 19.13 | 26.99 | 18.36 | 26.01 | 25.47 |
| ALH | 1.40 | 1.88 | 1.37 | 1.91 | 1.76 |

TABLE 2

| | Example 3 | | Example 4 | | Example 5 | | Example 6 | |
|---|---|---|---|---|---|---|---|---|
| | a first | a second | a first | a second | a first | a second | a first | a second |
| | groove | groove | groove | groove | groove | groove | groove | groove |
| MOT | 55.12 | 59.10 | 50.13 | 57.46 | 61.25 | 65.36 | 51.13 | 43.31 |
| VCL | 50.46 | 48.15 | 36.48 | 49.75 | 54.17 | 56.95 | 55.46 | 60.51 |
| VAP | 36.49 | 42.21 | 30.47 | 35.16 | 35.12 | 36.39 | 32.96 | 38.23 |
| VSL | 20.14 | 23.45 | 17.41 | 16.92 | 21.00 | 23.42 | 19.09 | 19.62 |
| ALH | 1.42 | 1.51 | 1.34 | 1.50 | 1.40 | 1.61 | 1.39 | 1.61 |

Referring to Table 1, it can be confirmed that in both Examples 1 and 2, sperm contained in the liquid collected from the second groove, which is the second groove away from the loading part, exhibited the most excellent characteristics such as motility and amplitude of lateral head displacement. In particular, compared to the first groove, the MOT was about 40% higher in Example 1 and 44% higher in Example 2, the VCL was about 44% higher in Example 1 and 42% higher in Example 2, respectively, the VAP was about 37% higher in both examples, the VSL was about 41% higher in both, and the ALH was about 34% higher in Example 1 and 39% higher in Example 2. These results indicate a statistically significant level of sperm selection.

In addition, in Example 2, it was further confirmed that the sperm contained in the liquid collected from the third groove generally exhibited better characteristics in terms of motility and amplitude of lateral head displacement compared to sperm collected from the first groove.

Furthermore, referring to Table 2, it can be seen that the differences in sperm characteristics between the first groove and the second groove were not as significant in Examples 3 to 6 compared to Examples 1 and 2. When comparing based on MOT and ALH: Example 3 showed about 7% (MOT) and 6% (ALH) difference, Example 4 showed about 14% (MOT) and 12% (ALH) difference, Example 5 showed about 6% (MOT) and 15% (ALH) difference, Example 6 showed-16% (MOT) and 15% (ALH) difference (values represent the ratio of the second groove to the first groove).

Although the embodiments have been described above, they are merely examples and not intended to limit the embodiments and it should be appreciated that various modifications and applications not described above may be made by one of ordinary skill in the art without departing from the essential features of the embodiment.

Therefore, it should be understood that the scope of the present invention includes changes, equivalents or substitutes of the technical spirit described above. For example, each component specifically shown in the embodiment of the present invention may be modified and implemented. In addition, it should be understood that differences related to these modifications and applications are within the scope of the present invention as defined in the appended claims.

1. A device for sorting sperm, comprising: a flow part configured to form an inclination,
wherein the flow part comprises at least two grooves spaced apart from each other along a direction of the inclination.

2. The device according to claim 1, wherein the device for sorting sperm is configured such that a liquid containing sperm flows along the flow part in an inclined direction.

3. A method of sorting sperm, the method comprising:
loading a sperm-containing liquid at a position on a flow part configured to form an inclination and provided with at least two grooves spaced apart from each other along a direction of the inclination; and
collecting only a liquid containing sperm that has flowed along the flow part and pooled in one or more of the plural grooves.

4. The method according to claim 3, wherein the collecting comprises: collecting the liquid in a groove that is not most adjacent to the loading position of the liquid.

5. The method according to claim 3, wherein the liquid flows along the flow part naturally by gravity without application of an external force.

6. The method according to claim 3, wherein a viscosity of the sperm-containing liquid to be loaded is prepared by mixing semen with one or more of physiological saline and a PBS buffer solution.
